(19) United States
(12) Patent Application Publication
(10) Pub. No.: US 2023/0060317 A1
(43) Pub. Date: Mar. 2, 2023

(54) COREGISTRATION OF MAGNETOENCEPHALOGRAPHY (MEG) DATA TO ANATOMICAL SPACE

(71) Applicant: FieldLine Inc., Boulder, CO (US)

(72) Inventors: Orang Alem, Erie, CO (US); Svenja Knappe, Boulder, CO (US)

(21) Appl. No.: 17/929,174

(22) Filed: Sep. 1, 2022

Related U.S. Application Data
(60) Provisional application No. 63/239,586, filed on Sep. 1, 2021.

(51) Int. Cl. A61B 5/245; G06T 17/20; G06T 7/33

(52) U.S. Cl. CPC A61B 5/245; G06T 17/20; G06T 7/344; G06T 2210/41; G06T 2207/30016; A61B 2562/0223

(57) ABSTRACT

Various embodiments comprise systems and methods to model the shape of a target subject to coregister an image generated by an on-subject sensor array to the anatomy of the subject. In some examples, a system constrains sensors to follow the contour of the target subject. The system generates a surface contour representation of the target subject based on the locations of the individual ones of the sensors. The system fits the surface contour representation of the target subject to an outer surface feature of an anatomical scan.

### RELATED APPLICATIONS

This Patent Application claims the benefit of and priority to U.S. Provisional Patent Application 63/239,586 entitled, “COREGISTRATION OF MAGNETOENCEPHALOGRAPHY (MEG) DATA TO ANATOMICAL SPACE” which was filed on Sep. 1, 2021, and which is hereby incorporated by reference in its entirety into this Patent Application.

### BACKGROUND

Images comprise a spatial distribution of information. Image coregistration is the process of transforming different sets of data into one coordinate system. In medicine, different imaging techniques are used to provide anatomic, metabolic, and functional information about the body. For multi-modal analysis, it is necessary to accurately coregister images from different medical imaging modalities that may have relatively different position and orientation coordinates. For example, data acquired from Magnetoencephalography (MEG) is routinely coregistered with Magnetic Resonance (MR) images to correlate functional information with anatomical structures. MEG systems image brain activity by detecting magnetic fields from neural currents using an array of magnetic sensors placed near the head of a subject and then computing the locations of the neural activity relative to the location of the sensor in a process referred to as source localization. The data from the sensors along with each sensor location is used to calculate the locations of neuronal signal sources to form MEG images of brain activity. For the source localization calculations, in addition to the data from the sensors, it is necessary to know the location and orientation of each sensor in a shared coordinate system. This coordinate system is the MEG or sensor coordinate frame.

Some MEG systems have sensors that can move independently and conform to the size and shape of the head. These MEG systems are referred to as on-scalp or conformal MEG. The sensors used in conformal MEG may comprise Optically-Pumped Magnetometers (OPMs), however other types of sensors can also be used for on-scalp or conformal MEG. For on-scalp MEG systems, the location and orientation information for the sensor array is determined for every subject and every time the sensors are placed on the scalp. Unfortunately, it is difficult to coregister MEG data generated in conformal MEG systems with anatomical data.

### OVERVIEW

This Overview is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Various embodiments of the present technology relate to solutions for magnetic field sensing and generation. Some embodiments comprise a method to model the shape of a target subject to coregister an image generated by an on-subject sensor array to the anatomy of the subject. The method comprises constraining sensors to follow the contour of the target subject. The method further comprises generating a surface contour representation of the target subject based on the locations of the individual ones of the sensors. The method further comprises fitting the surface contour representation of the target subject to an outer surface feature of an anatomical scan.

Some embodiments comprise an apparatus to model the shape of a target subject to coregister an image generated by an on-subject sensor array to the anatomy of the subject. The apparatus comprises a sensor mount and a computing device. The computing device comprises a processing system, memory, and program instructions stored on the memory that, when executed by the processing system, drive the processing system to perform operations. The sensor mount is configured to constrain sensors to follow a contour of the target subject. The processing system generates a surface contour representation of the target subject based on the locations of the individual ones of the sensors. The processing system fits the surface contour representation of the target subject to an outer surface feature of an anatomical scan.

Some embodiments comprise a system to model the shape of a target subject to coregister an image generated by an on-subject sensor array to the anatomy of the subject. The system comprises a computing device comprising a processing system, one or more computer-readable storage media, and program instructions stored on the one or more computer-readable storage media that, when executed by the processing system, direct the processing system to perform operations. The operations comprise receiving location information for sensors in the on-subject sensor array that characterizes the locations of individual ones of the sensors. The sensors are constrained to follow the contour of the target subject. The operations further comprise generating a surface contour representation of the target subject based on the locations of the individual ones of the sensors. The operations further comprise fitting the surface contour representation of the target subject to an outer surface feature of an anatomical scan.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary Magnetoencephalography (MEG) system.

FIG. 2 illustrates an exemplary MEG system.

FIG. 3 illustrates an exemplary coregistration process.

FIG. 4 illustrates an exemplary sensor mount.

FIG. 5 illustrates an exemplary coregistration process.

FIG. 6 illustrates an exemplary computing apparatus according to some embodiments.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### TECHNICAL DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

The examples herein present systems and methods to coregister MEG data with anatomical data based on sensor location in a conformal MEG. In conformal MEG, the sensors, like Optically Pumped Magnetometers (OPMs), conform to the shape a target subject (e.g., a human head). By conforming to the shape of the target subject, the location and orientation of the sensors may be used to define a 3-Dimentional (3D) boundary or contour of the surface geometry of the target. For example, the locations of the sensors when conformed to the surface of the human had may be used to create a surface contour depicting the scalp. Algorithmic techniques can be used to determine or model the surface representation of the target head based on the sensor locations. Some algorithmic techniques include point-based modeling algorithms that build a 3D representation of a surface using points near the surface and projection operators. Other algorithmic techniques generate a surface mesh representing the boundary of the head surface. As the number of sensors used in the conformal MEG increases, the detail of the surface representation of the head surface increases. For example, a sensor array may cover a large area of the head, or an area of the head may comprise a high sensor density. The 3D surface representation is used to align or coregister the MEG images with other anatomical images like Magnetic Resonance Imaging (MRI) scans that have differing coordinate systems. The systems and methods described herein may not require any addition methods for the registration of medical images, such as optical scanning, dipole localization using HPI coils, or other forms of co-registration techniques. Now turning to the Figures.

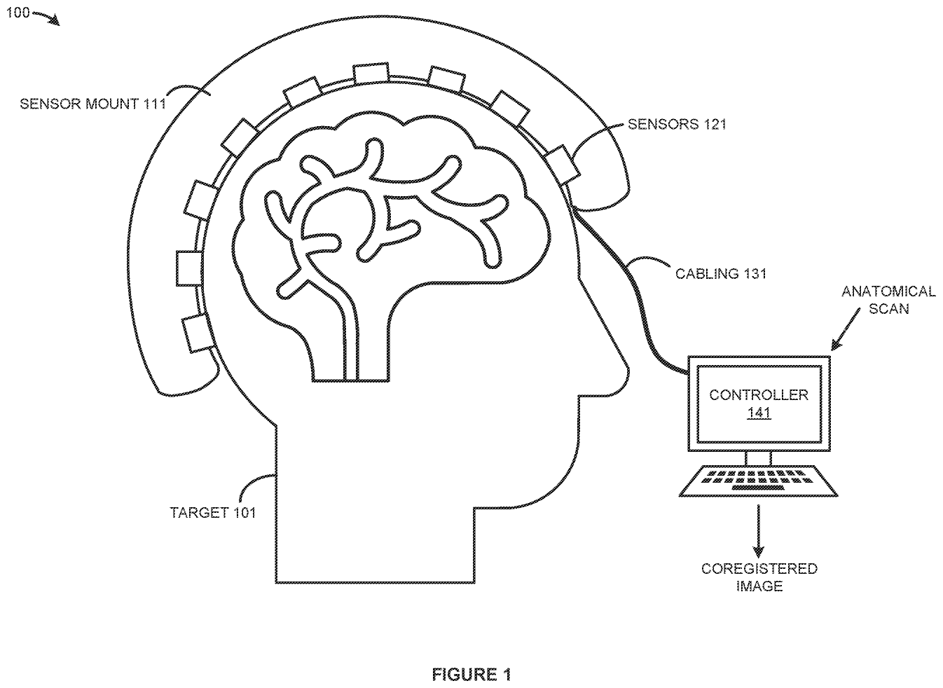

FIG. 1 illustrates Magnetoencephalography (MEG) system 100. MEG system 100 performs operations like detecting magnetic fields and relating the detecting magnetic fields to neuronal activity for use in medical applications. Exemplary medical applications include identifying brain activity and diagnosing conditions like stroke, epilepsy, neuronal injuries, neuronal disorders, and/or other types of medical conditions relating to brain/neuron activity. MEG system 100 comprises target 101, sensor mount 111, sensors 121, cabling 131, and controller 141. In other examples, MEG system 100 may differ. In this example, target 101 comprises a human head however target 101 may comprise any magnetic field source including a non-biological magnetic field source.

Sensor mount 111 comprises a wearable apparatus that secures the position and orientation of sensors 121 in locations proximate to target 101. For example, sensor mount 111 may securely adhere sensors 121 to the scalp of target 101. Sensor mount 111 may comprise a rigid helmet or a flexible cap. In the example where sensor mount 111 comprises a flexible cap, the flexible cap may comprise an elastic material like rubber and is placed on the head of target 101. The flexible cap forms naturally to the shape of target 101 and compresses the sensors onto the scalp of target 101 to fix in place both the position and orientation of sensors 121. In the case where sensor mount 111 comprises a rigid helmet, the rigid helmet may comprise slots to hold the sensors. The slots form channels that fix in place both the position and orientation of sensors 121. The rigid helmet is placed on the head of target 101 and sensors 121 move through their respective channels to contact the surface of target 101. For example, the rigid helmet may comprise ratchet mechanisms, pneumatic mechanisms, set screws, springs, and/or another type of mechanism to move sensors 121 through the slots to contact target 101 and hold sensors 121 in place once in contact. In either example, sensor mount 111 conforms sensors 121 to the surface geometry of target 101.

Sensors 121 comprise magnetometers that sense magnetic fields generated by a magnetic field source in target 101 and generate signals that characterize the strength of the detected magnetic field. In this example, the magnetic field source comprises the brain of target 101. The neuronal activity in the brain of target 101 comprises intercellular electromagnetic signals. Sensors 121 sense the magnetic component of the electromagnetic signals to detect the neuronal activity. Sensors 121 form a sensor array that is contoured to the head of target 101 by sensor mount 111. In some examples, MEG system 100 does not include sensor mount 111 and sensors 121 may be contoured to the head of target in another way. For example, sensors 121 may be directly adhered to the scalp of target 101 using tape, glue, or another type of temporary adhesive to form an on-scalp sensor array. Exemplary magnetometers that may comprise sensors 121 include Optically Pumped Magnetometers (OPMs), atomic magnetometers, gradiometers, nitrogen vacancy centers, high-temperature Superconducting Quantum Interference Devices (SQUIDs), and the like. Sensors 121 are coupled to controller 141 over cabling 131. Cabling 131 comprises sheathed metallic wires. For example, sensors 121 may transfer signaling that characterizes the sensed magnetic field to controller 141 over cabling 131. In some examples, cabling may be replaced with, or used in addition with, a wireless transceiver system (e.g., antennas) to transfer communications between controller 141 and sensors 121 over a wireless networking protocol like bluetooth.

Controller 141 is representative of one or more computing devices configured to drive the operation of sensors 121 and to coregister the locations of sensors 121 to anatomical scans of target 101 like Magnetic Resonance Imaging (MRI) scans to relate neuronal activity detected by sensors 121 to anatomical structures of the brain. For example, the anatomical scan may depict the prefrontal cortex of target 101 and the coregistration may correlate detected neuron activity to the physical structure of the prefrontal cortex. The one or more computing devices comprise processors, memories, and transceivers, that are connected over bus circuitry. The processors may comprise Central Processing Units (CPUs), Graphical Processing Units (GPUs), Application Specific Integrated Circuits (ASICs), Field Programmable Gate Arrays (FPGAs), and the like. The memories may comprise Random Access Memory (RAM), flash circuitry, Solid States Drives (SSDs), Hard Disk Drives (HDDs), and the like. The memory stores software like operating systems, MEG applications, coregistration applications, sensor data, and the like. The processors retrieve and execute the software from the memory to drive the operation of controller 141.

Controller 141 transfers instructions to sensors 121 that direct sensors 121 to measure a magnetic field generated by neuronal activity in target 101 over cabling 131. Controller 141 receives sensor data from sensors 121 that characterizes the strength of the sensed magnetic field. The sensor data may be addressed (e.g., sensor ID) to correlate the measured magnetic field strengths with individual ones of sensors 121. Controller 141 executes a MEG application to generate a MEG image based on the magnetic field strengths received from sensors 121. The MEG image depicts the magnetic field detected by sensors 121 in three dimensions to illustrate the neuronal activity in the brain of target 101.

Controller 141 generates a surface contour of the target based on the location of sensors 121. For example, controller 141 may execute a coregistration application to generate the surface contour based on the sensor locations. When sensors 121 contact the surface of target 101, their spatial locations correspond to geometric points on the surface of target 101. The surface contour generated from these geometric points provides an anatomically accurate map of the target surface. The surface contour characterizes the surface geometry of target 101 and may comprise a Three-Dimensional (3D) mesh, a 3D surface illustration, and/or some other type of 3D representation of the target's surface geometry. Controller 141 coregisters the MEG image of neural activity generated using sensors 121 to an anatomical brain scan of target 101 to relate measured brain activity to a specific region of the brain. These anatomical images depict the anatomy of the head surface and the anatomy of the brain inside. By aligning and fitting the surface contour of target 101 to the anatomical scan of target 101, the overlayed image correlates MEG images to the anatomically accurate regions of the brain. The anatomical brain scan may comprise a Magnetic Resonance Imaging (MRI) scan, Computer Topography (CT) scan, or some other type of brain image. The sensor location and orientation information can be determined optically, mechanically, electrically, magnetically, and/or by another sensor localization technique.

Although the above examples are discussed with relation to Magnetoencephalography (MEG), other magnetic imaging modalities are contemplated herein. For example, MEG system 100 may instead comprise a Magnetocardiography (MCG) system, a Magnetogastrography (MGG) system, a Magnetomyography (MMG) system, or another type of anatomical magnetic sensing technology.

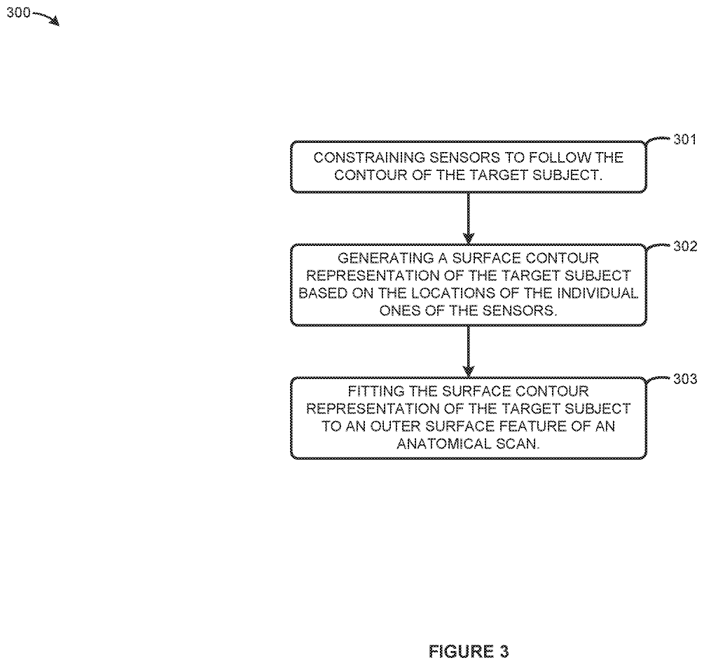

In some examples, MEG system 100 implements process 300 described in FIG. 3. It should be appreciated that the structure and operation of MEG system 100 may differ in other examples.

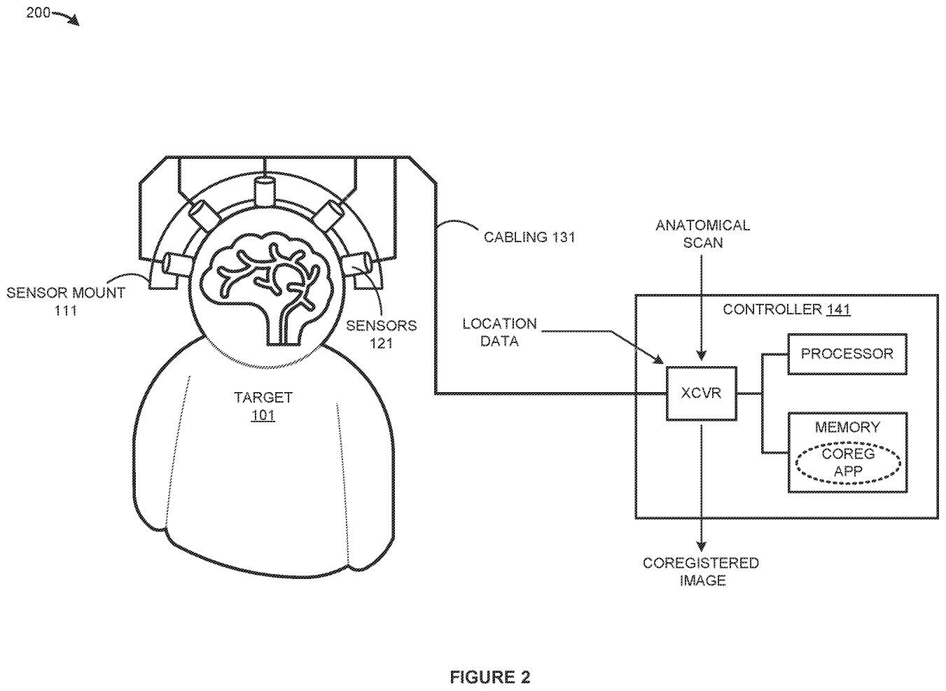

FIG. 2 illustrates environment 200. Environment 200 comprises a schematic view of MEG system 100 illustrated in FIG. 1. Environment 200 comprises target 101, sensor mount 111, sensors 121, cabling 131, and controller 141. Controller 141 is illustrated comprising transceiver (XCVR) circuitry, a processor, and a memory connected over bus circuitry. The memory stores a coregistration application (COREG APP). Controller 141 typically comprises additional components like user interface systems and a power supply, however the additional components are omitted for the sake of clarity.

In operation, sensor mount 111 is worn by target 101. Sensors 121 are embedded into sensor mount 111. Sensors 121 are securely pressed against the scalp of target 101 by sensor mount 111 to conform sensors 121 to the surface contour of target. For example, sensor mount 111 may comprise a flexible material that, when worn by target 101, conforms to the shape of the head of target 101 and compresses sensors 121 against the surface of the head to follow the contour of target 101. Sensors 121 measure a magnetic field generated by the brain of target 101 and transfer signaling to controller 141 characterizing the detected field. The processing circuitry of controller 141 receives the sensor data via the transceiver circuitry and generates a MEG image based on the data. The MEG image depicts brain activity detected by sensors 121. Additionally, the MEG image comprises a coordinate system that defines the location of the detected brain activity.

Controller 141 receives location data for sensors 121 locations via the transceiver circuitry. The location data comprises spatial locations for individual ones of sensors 121. The spatial locations of the sensors may be determined in a variety of ways like optical, mechanical, electromagnetic sensing. Electromagnetic sensing comprises the detection of an electric field, the detection of a magnetic field, or a combination thereof to determine the location of sensors 121. In some examples, sensor mount 111 comprises embedded coils at known locations that produce an electromagnetic field in response to electric current. Sensors 121 measure the electromagnetic field produced by the embedded coils and report the measured field strengths to controller 141. Controller 141 may correlate the measured field strengths to distances between sensors 121 and the known locations of the embedded coils to determine the spatial locations of the sensors. In some examples, sensor mount 111 may comprise set screws with known locations that move sensors 121 to conform to the shape of target 101 and fix the orientation and location of sensors 121. Controller 141 may receive set screw data that indicates how far the set screws have moved sensors 121 to determine the spatial locations of sensors 121. In some examples, sensor mount 111 may comprise scannable fiduciary marks at multiple surface locations that define its location and orientation. Controller 141 may receive the scanned data and responsively determine the spatial locations of sensors 121. In some examples, cameras (not illustrated), may image sensors 121 from multiple orientations. Controller 141 may ingest the images and determine the spatial locations of sensors 121 based on the image data. In some examples, a scanner may scan Radio Frequency (RF) IDs of sensors 121 and transfer the scanned data to controller 141. Controller 141 may process the RF data to determine the spatial locations of sensors 121. In some examples, the aforementioned sensor localization techniques may be combined to determine the spatial locations of the sensors or other sensor localization techniques may be used. In some examples, the spatial locations of sensors 121 may already be known to controller 141 and controller 141 may retrieve the spatial locations from memory.

In some examples, additional markers (not illustrated) may be places on the surface of target 101 to more fully characterize the surface geometry of target 101. The markers may comprise magnetic markers like coils. The coils may be localized by the sensors 121 with respect to the geometry of sensors 121. The markers may be placed in locations not covered by the sensor array formed by sensors 121. In particular, the markers may be placed in unique locations on target 101 to clearly define the geometry of target 101 and aid in overlaying the sensor geometry with the anatomy of target 101 during image coregistration. In some examples, the markers may comprise small head position indicator coils that are placed on unique features of the subject to extend the coverage of the locations around the head. The coils are then localized by the sensor array formed by sensors 121. The locations of the position indicator coils in addition to the sensor locations are used to coregister sensors 121 with the anatomy of target 101. Placing the marker coils in positions that can be easily identified in the anatomical image aids the coregistration.

The processing circuitry generates a 3D surface contour of target 101 based on the spatial locations of the sensors 121. The spatial locations of sensors 121 correlate to geometric points on the surface of target 101, and controller 141 may generate a surface contour based on the geometric points. The processing circuitry may implement one or more algorithmic techniques to determine and model the surface representation of target 101 based on the spatial locations of sensor 121. For example, the processing circuitry may execute a point-based modeling algorithm that receives spatial locations as inputs and outputs a 3D representation of the surface of target 101 using points near the surface and projection operators. The processing circuitry may execute a surface mesh algorithm that receives the spatial locations as inputs and outputs a surface mesh representing the boundary of the head surface. The algorithmically generated surface contour characterizes the surface geometry of target 101. In particular, the surface contour defines points in a MEG coordinate system that are co-located with the outer surface of target 101. The surface points in the MEG coordinate system are used to determine additional points in the MEG coordinate system co-located with the sensed brain activity of target 101. The surface contour may comprise a 3D mesh, a 3D surface illustration, and/or some other type of 3D representation of target 101′s surface geometry.

Controller 141 receives an anatomical scan from an external system via the transceiver circuitry. For example, the controller 141 may receive an MRI scan of the head of target 101 from a patient database that stores medical information. The anatomical scan comprises a different coordinate system than the MEG image. The anatomical coordinate system defines the physical locations of brain structures in target 101 depicted in the scan. The processing circuitry coregisters the surface contour of target 101 with the anatomical scan to generate an overlayed image. In some examples, the anatomical scan may depict an outer surface feature of target 101. The processing circuitry retrieves and executes the coregistration application from memory. The coregistration application aligns the surface contour of target 101 with the outer surface feature of target 101 depicted in the anatomical scan to fit the surface contour to the anatomical scan and generate the overlayed image. The overlayed image relates brain anatomy depicted by the anatomical scan to the surface geometry of the target depicted by the surface contour. In particular, the overlayed image coregisters the coordinate system of the anatomical data with the coordinate system of the MEG image. The coregistration creates a one-to-one relationship between points in the anatomical space to points in the MEG space. In doing so, controller 141 relates detected brain activity from sensors 121 to the physical brain structure depicted by the anatomical scan. Controller 141 may transfer the coregistered image to downstream systems.

FIG. 3 illustrates process 300. Process 300 is representative of an image coregistration process to coregister MEG images with anatomical images based on the spatial locations of sensors. Portions of process 300 may be implemented in program instructions in the context of any of the hardware components, software applications, module components, or other such elements of one or more computing devices.

The operations of process 300 comprises constraining sensors to follow the contour of the target subject (step 301). The operations further comprise generating a surface contour representation of the target subject based on the locations of the individual ones of the sensors (step 302). The operations further comprise fitting the surface contour representation of the target subject to an outer surface feature of an anatomical scan (step 303).

Referring back to FIGS. 1 and 2, MEG system 100 includes a brief example of process 300 as implemented by the various hardware and software components that comprise system 100. The structure and operation of MEG system 100 may differ in other examples.

In operation, sensor mount 111 is worn by target 101. Sensor mount 111 constrains sensors 121 to follow the contour of target 101 (step 301). Sensor mount 111 constrains each of sensors 121 in both position and orientation onto the scalp of target 101. For example, sensor mount 111 may comprise a rigid helmet with slots to hold sensors 121. The slots physically constrain the position and orientation sensors 121. Controller 141 determines the spatial locations of sensors 121. The spatial locations of sensors 121 indicate their locations on the scalp of target 101 in relation to one another. For example, sensor mount 111 may comprise a magnetic dipole with a fixed location and controller 141 may receive magnetic dipole strengths from sensors 121 and determine the spatial locations based on the measured strengths. In some examples, the spatial locations of sensors 121 are already known and controller 121 may simply retrieve the locations from memory.

Controller 141 generates a surface contour of target 101 based on the spatial locations of sensors 121 (step 302). For example, controller 141 may plot the spatial locations of sensors 121 in the MEG image coordinate system to determine the relative locations of sensors 121 on target 101. Controller 141 uses plotted locations to generate a 3D surface mesh that depicts the head shape of target 101. Controller 141 accesses an anatomical scan (e.g., MRI scan) of target 101. The anatomical scan depicts the brain anatomy and an outer surface of target 101. The anatomical scan comprises a different coordinate system than the MEG image. Controller 141 fits the surface contour of target 101 to the outer surface feature of target 101 depicted in the anatomical scan (step 303). For example, controller 141 may overlay and resize the surface contour onto the anatomical scan and position the surface contour to align with the outer surface feature depicted by the scan. The alignment creates a shared coordinate system between the coordinate system in the MEG image and the coordinate system in the anatomical scan. By fitting the surface contour of target 101 to the anatomical scan, controller 141 correlates MEG data depicted by the MEG image to anatomical data depicted in the anatomical scan.

Advantageously, MEG system 100 effectively constrains sensors 121 to the surface of target 101 and determines the surface geometry of target 101 based on the locations of sensors 121. Moreover, MEG system 100 efficiently correlates anatomical brain scans with a surface contour of target 101 to coregister the coordinate system of the anatomical scan to the coordinate system of a MEG image. By coregistering the coordinate systems, MEG system 100 may relate brain activity detected in the MEG image to anatomical structures depicted in the anatomical scan.

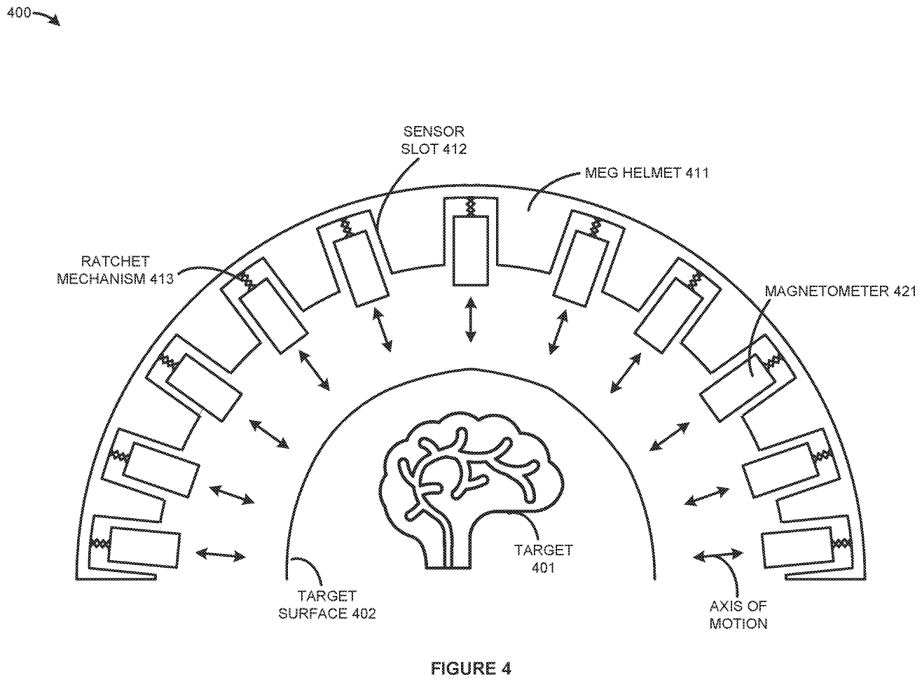

FIG. 4 illustrates environment 400 to constrain magnetometers onto a target subject. Environment 400 comprises target 401, target surface 402, and MEG helmet 411. Environment 400 illustrates a cross-sectional view of a sensor mount in accordance with the various examples described herein. MEG helmet 411 is representative of a rigid sensor mount and is an example of sensor mount 111 illustrated in FIGS. 1 and 2, however mount 111 may differ. MEG helmet 411 comprises sensor slots 412, ratchet mechanisms 413, and magnetometers 421. Magnetometers 421 may comprise OPMs, atomic magnetometers, gradiometers, nitrogen vacancy centers, SQUIDs, and/or other types of magnetometers. Target 401 is representative of a human brain and target surface 402 is representative of the outer surface of target 401 (e.g., a human scalp).

MEG helmet 411 is worn by target 401 and comprises a rigid sensor mount configured to provide structural support to a sensor array formed by magnetometers 421. MEG helmet 411 may be constructed from a rigid plastic or other materials that do not inhibit magnetic sensing of a magnetic field generated by neuronal activity in target 401. Magnetometers 421 reside in slots 412. Slots 412 comprises indented regions in helmet 411 shaped to house magnetometers 421. For example, if magnetometers 421 are cylindrically shaped, slots 412 may comprise cylindrically shaped indentations that correspond to the shape and size of magnetometers 421. Slots 412 constrain the orientation, location, and axis of motion of magnetometers 421 as illustrated in FIG. 4.

Magnetometers 421 are coupled to MEG helmet 411 via ratchet mechanisms 413 and are housed in slots 412. Ratchet mechanisms 413 may comprise set screws, springs, pneumatics, and/or other mechanical systems configured to move magnetometers 421 along their respective axes of motion and lock the location of magnetometers 421 in place once in contact with target surface 402. Ratchet mechanisms 413 are adjusted to move magnetometers 421 through slots 412 along their respective axes of motion until in contact with target surface 402. Once in contact with target surface 402, ratchet mechanisms 413 and slots 412 constrain the position and orientation of magnetometers 421 to follow the surface contour of target surface 402. Since the locations of slots 412 are fixed and their locations are known, the distance that magnetometers 421 move through slots 412 to contact target surface 402 can be used to identify the spatial locations of magnetometers 421. For example, the set screw setting of one of ratchet mechanisms 413 may be used to determine the spatial locations of the corresponding one of sensors 421.

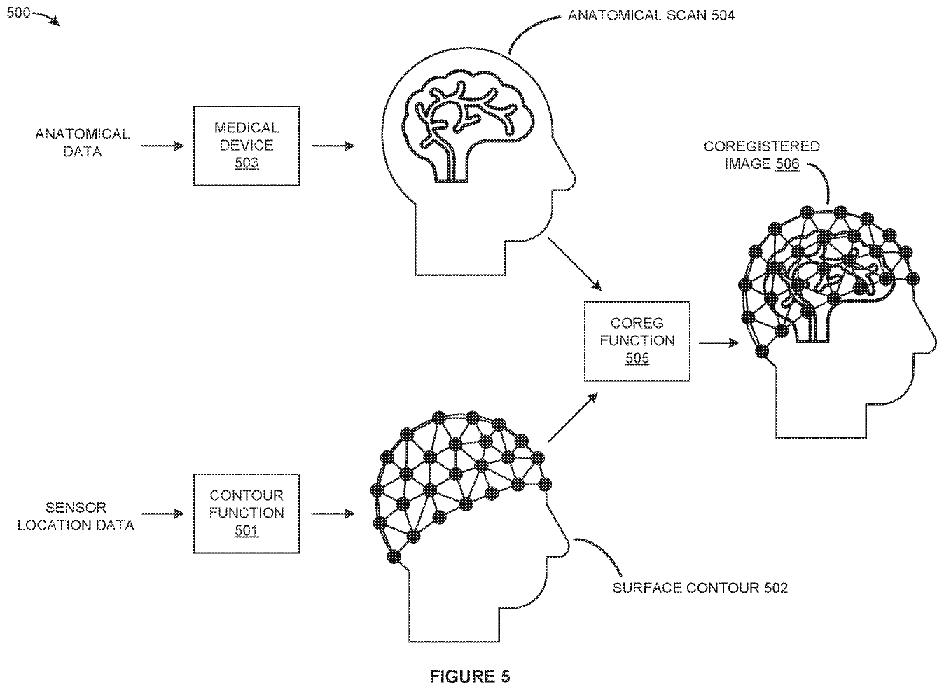

FIG. 5 illustrates process 500. Process 500 comprises a block diagram representative of a process to coregister MEG data with anatomical data. Process 500 is an example of process 300 illustrated in FIG. 3, however process 300 may differ. In operation, contour function 501 receives sensor location data. The sensor location data defines the spatial locations of magnetometers like OPMs on the scalp of a target subject. Contour function 501 applies the spatial locations of the sensors to a MEG coordinate system to relate the locations of the magnetometers to one another. The MEG coordinate system additionally defines the location of neuronal activity in the target subject depicted in a MEG image. Contour function 501 generates a 3D mesh of the outer surface of the target subject based on the plotted coordinates for the magnetometers. Contour function 501 outputs the 3D mesh as surface contour 502. Surface contour 502 comprises points and interconnecting line segments that represent the surface geometry of the target subject. Surface contour 502 is a 3D representation of the surface geometry of the target subject but is illustrated in two dimensions for the sake of clarity.

Medical device 503 receives anatomical data and generates anatomical scan 504. Anatomical scan 504 depicts the brain anatomy and an outer surface feature of the target subject. Anatomical scan 504 may comprise an MRI image, CT scan, or some other type of brain image. Medical device 503 is representative of an MRI machine or other type of medical device configured to generate anatomical scan 504. Anatomical scan 504 comprises an anatomical coordinate system that defines the locations of brain structures depicted in scan 504.

Coregistration (COREG) function 505 ingests surface contour 502 and anatomical scan 504. Coregistration function 505 locates the outer surface feature of the target subject depicted in anatomical scan 504. Coregistration function 505 aligns surface contour 502 with the outer surface feature in anatomical scan 504 to fit contour 502 to scan 504. Once aligned, coregistration function 505 overlays surface contour 502 onto anatomical scan 504 to generate coregistration image 506. Coregistered image 506 relates the anatomical coordinate system of scan 504 to the MEG coordinate system used in surface contour 502. In doing so, coregistered image 506 is used to identify anatomical structures of the brain depicted by scan 504 and corresponding neuronal activity depicted by the MEG scan related to surface contour 502. In some examples, this relation can be used to select sensors to measure the magnetic field emitted by a region of interest in the target subject's brain. Contour function 501 and coregistration function 505 comprise examples of the coregistration function implemented by controller 141 illustrated in FIG. 2.

In some examples, process 500 may include additional coregistration techniques to improve the accuracy of coregistered image 506. For example, a digitizer pen may be used, in addition to sensor coregistration, to model the surface geometry of the target subject. A digitizer pen is a type of medical device to model surface geometry. The digitizer pen contacts the surface of the target subject at multiple points. The digitizer pen records the contacts and plots the contacted points in a three-dimensional space to build an additional surface contour of the target subject. Coregistration function 505 receives anatomical scan 504, surface contour 502, and the additional surface contour generated by the digitizer pen. Coregistration function 505 aligns surface contour 502 and the additional surface contour with the outer surface feature depicted by scan 504. Once aligned, coregistration function 505 overlays surface contour 502 and the additional surface contour onto anatomical scan 504 to generate coregistration image 506. In doing so, coregistration function 505 improves the accuracy of surface contour 502 and may model additional features of the target subject (e.g., the face) where sensors are not positioned.

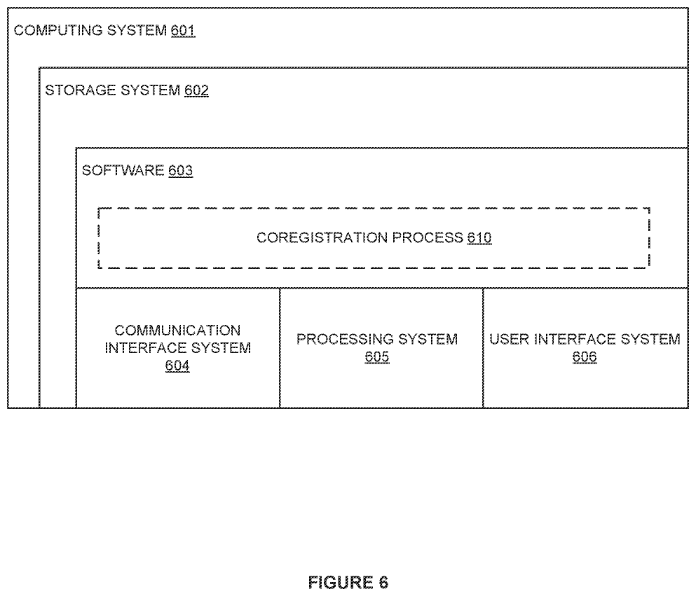

FIG. 6 illustrates computing system 601 according to an implementation of the present technology. Computing system 601 is representative of any system or collection of systems in which the various processes, programs, services, and scenarios disclosed herein for coregistering anatomical scans and MEG scans may be implemented. For example, computing system 601 may be representative of controller 141 and/or any other computing device contemplated herein. Examples of computing system 601 include, but are not limited to, computers, servers, network controllers, web servers, and cloud computing platforms, as well as any other type of physical or virtual server machine, physical or virtual router, container, and any variation or combination thereof. Computing system 601 may be implemented as a single apparatus, system, or device or may be implemented in a distributed manner as multiple apparatuses, systems, or devices. Computing system 601 includes, but is not limited to storage system 602, software 603, communication interface system 604, processing system 605, and user interface system 606. Processing system 605 is operatively coupled with storage system 602, communication interface system 604, and user interface system 606.

Processing system 605 loads and executes software 603 from storage system 602. Software 603 includes and implements coregistration process 610, which is representative of the MEG/anatomical coregistration processes discussed with respect to the preceding Figures. When executed by processing system 605, software 603 directs processing system 605 to operate as described herein for at least the various processes, operational scenarios, and sequences discussed in the foregoing implementations. Computing system 601 may optionally include additional devices, features, or functionality not discussed here for purposes of brevity.

Processing system 605 may comprise a micro-processor and other circuitry that retrieves and executes software 603 from storage system 602. Processing system 605 may be implemented within a single processing device but may also be distributed across multiple processing devices or sub-systems that cooperate in executing program instructions. Examples of processing system 605 include general purpose CPUs, GPUs, DSPs, ASICs, FPGAs, and logic devices, as well as any other type of processing device, combinations, or variations thereof.

Storage system 602 may comprise any computer readable storage media that is readable by processing system 605 and capable of storing software 603. Storage system 602 may include volatile and nonvolatile, removable, and non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 602 may also include computer readable communication media over which at least some of software 603 may be communicated internally or externally. Storage system 602 may be implemented as a single storage device but may also be implemented across multiple storage devices or sub-systems co-located or distributed relative to each other. Storage system 602 may comprise additional elements, such as a controller, capable of communicating with processing system 605 or possibly other systems.

Software 603 (coregistration process 610) may be implemented in program instructions and among other functions may, when executed by processing system 605, direct processing system 605 to operate as described with respect to the various operational scenarios, sequences, and processes illustrated herein. For example, software 603 may include program instructions for generating a surface mesh of a target subject based on magnetometer spatial locations and fitting the surface mesh to an anatomical scan to coregister MEG data to anatomical data.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 603 may include additional processes, programs, or components, such as operating system software, virtualization software, or other application software. Software 603 may also comprise firmware or some other form of machine-readable processing instructions executable by processing system 605.

In general, software 603 may, when loaded into processing system 605 and executed, transform a suitable apparatus, system, or device (of which computing system 601 is representative) overall from a general-purpose computing system into a special-purpose computing system customized to coregister anatomical data and MEG data. Indeed, encoding software 603 on storage system 602 may transform the physical structure of storage system 602. The specific transformation of the physical structure may depend on various factors in different implementations of this description. Examples of such factors may include, but are not limited to, the technology used to implement the storage media of storage system 602 and whether the computer-storage media are characterized as primary or secondary storage, as well as other factors.

For example, if the computer readable storage media are implemented as semiconductor-based memory, software 603 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 604 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 601 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

While some examples provided herein are described in the context of computing devices for generating surface contours based on sensor locations to coregister MEG images and anatomical scans, it should be understood that the systems and methods described herein are not limited to such embodiments and may apply to a variety of other MEG environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number, respectively. The word “or” in reference to a list of two or more items, covers all of the following interpretations of the word: any of the items in the list, all of the items in the list, and any combination of the items in the list.

The phrases “in some embodiments,” “according to some embodiments,” “in the embodiments shown,” “in other embodiments,” and the like generally mean the particular feature, structure, or characteristic following the phrase is included in at least one implementation of the present technology and may be included in more than one implementation. In addition, such phrases do not necessarily refer to the same embodiments or different embodiments.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a method claim, other aspects may likewise be embodied as a computer-readable medium claim, or in other forms, such as being embodied in a means-plus-function claim. Any claims intended to be treated under 35 U.S.C. § 112(f) will begin with the words “means for” but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. A method to model the shape of a target subject to coregister an image generated by an on-subject sensor array to the anatomy of the subject, the method comprising:
constraining sensors of the on-subject sensor array to follow a contour of the target subject;
generating a surface contour representation of the target subject based on locations of the individual ones of the sensors; and
fitting the surface contour representation of the target subject to an outer surface feature of an anatomical scan.

2. The method of claim 1 wherein the sensors of the on-subject sensor array are constrained to follow a scalp of the target subject for Magnetoencephalography (MEG).

3. The method of claim 1 wherein the sensors comprise magnetometers.

4. The method of claim 1 wherein the sensors comprise gradiometers.

5. The method of claim 1 wherein the sensors comprise Optically-Pumped Magnetometers (OPMs).

6. The method of claim 1 wherein the sensors comprise nitrogen-vacancy centers.

7. The method of claim 1 wherein the sensors comprise high-temperature Superconducting Quantum Interference Devices (SQUIDs).

8. The method of claim 1 wherein constraining the sensors comprises ratcheting the individual ones of the sensors to contact an outer surface of the target subject to follow the contour of the target subject.

9. The method of claim 1 wherein constraining the sensors comprises compressing the individual ones of the sensors against an outer surface of the target subject to follow the contour of the target subject and to fix the orientation and position of the individual ones of the sensors.

10. The method of claim 1 further comprising determining the locations of the sensors by mechanical constraining.

11. The method of claim 1 further comprising determining the locations of the sensors by electromagnetic sensing.

12. The method of claim 1 further comprising determining the locations of the sensors by optical sensing.

13. The method of claim 1 wherein generating the surface contour representation of the target subject comprises generating the surface contour representation of the target subject based on the locations of the individual ones of the sensors and additional locations of a set of one or more markers attached to a surface of the target subject.

14. An apparatus to model the shape of a target subject to coregister an image generated by an on-subject sensor array to the anatomy of the subject, the apparatus comprising:
a sensor mount configured to constrain sensors of the on-subject sensor array to follow a contour of the target subject; and
a computing device comprising a processing system, memory, and program instructions stored on the memory, that, when executed by the processing system, direct the processing system to:
generate a surface contour representation of the target subject based on locations of the individual ones of the sensors; and
fit the surface contour representation of the target subject to an outer surface feature of an anatomical scan.

15. The apparatus of claim 14 wherein the sensors of the on-subject sensor array are constrained to follow a scalp of the subject for Magnetoencephalography (MEG).

16. The apparatus of claim 14 wherein the sensors comprise magnetometers.

17. The apparatus of claim 14 wherein the sensors comprise gradiometers.

18. The apparatus of claim 14 wherein the sensors comprise Optically-Pumped Magnetometers (OPMs).

19. The apparatus of claim 14 wherein the sensors comprise nitrogen-vacancy centers.

20. The apparatus of claim 14 wherein the sensors comprise high-temperature Superconducting Quantum Interference Devices (SQUIDs).

21. The apparatus of claim 14 further comprising:
a set of a set of one or more markers attached to a surface of the target subject at additional locations; and wherein:
the computing device comprising the processing system, memory, and program instructions stored on the memory, that, when executed by the processing system, further direct the processing system to:
generate the surface contour representation of the target subject based on the locations of the individual ones of the sensors and the additional locations of the set of one or more markers attached to the surface of the target subject.

22. A system to model the shape of a target subject to coregister an image generated by an on-subject sensor array to the anatomy of the subject, the system comprising:
a computing device comprising a processing system, one or more computer-readable storage media, and program instructions stored on the one or more computer-readable storage media that, when executed by the processing system, direct the processing system to perform operations comprising:
receiving location information for sensors of the on-subject sensor array that characterizes locations of individual ones of the sensors wherein the sensors of the on-subject sensor array are constrained to follow a contour of the target subject;
generating a surface contour representation of the target subject based on the locations of the individual ones of the sensors; and
fitting the surface contour representation of the target subject to an outer surface feature of an anatomical scan.
